(19) United States
(12) Patent Application Publication
(10) Pub. No.: US 2026/0013966 A1
(43) Pub. Date: Jan. 15, 2026

(54) MEDICAL DEVICE PACKAGE WITH FIRST USE INDICATOR LABEL

(71) Applicant: Hollister Incorporated, Libertyville, IL (US)

(72) Inventors: Vivienne McNulty, Ballina (IE); Shane O’Malley, Ballina (IE); Michelle Heffernan, Sligo (IE)

(21) Appl. No.: 19/335,787

(22) Filed: Sep. 22, 2025

Related U.S. Application Data
(63) Continuation of application No. 18/043,234, filed on Feb. 27, 2023, now Pat. No. 12,433,709.
(60) Provisional application No. 63/074,596, filed on Sep. 4, 2020.

(51) Int. Cl. A61B 50/30

(52) U.S. Cl. CPC A61B 50/30; A61B 2560/02

(57) ABSTRACT

The present disclosure is directed to a medical product package that may comprise a case for receiving a medical product, the case having an open end; a cap for sealing the open end of the case; a hinge having one end connected to the cap and a second end connected to the case; an indentation in the outer surface of the cap, the indentation having a reduced outer dimension compared to the rest of the outer surface of the cap in which it is formed; an indentation in an outer surface of the case, the indentation having a reduced outer dimension compared to the rest of the outer surface of the case in which it is formed, the indentation in the outer surface of the cap and the indentation in the outer surface of the case being adjacent to each other when the cap is in a fully closed position; and a first use indication label affixed to the cap and case and spans both the indentation in the cap and the indentation in the case, wherein the first use indication label is made of a material that does not tear upon opening of the package.

### CROSS-REFERENCE TO RELATED APPLICATIONS

The present is a continuation application of U.S. application Ser. No. 18/043,234, filed on Feb. 27, 2023, which is the US National Stage Application of PCT Application No. PCT/US2021/048635, filed Sep. 1, 2021, which claims the benefit of and priority to U.S. Provisional Patent Application Ser. No. 63/074,596, filed Sep. 4, 2020, the content of which applications are incorporated herein by reference.

### FIELD OF THE DISCLOSURE

The present disclosure is directed to medical device packages that provide an indication that the package has been previously opened. More particularly, the present disclosure is directed to a medical device package that includes a first use indicator label that assumes a configuration after the package is re-closed or re-sealed. Even more particularly, the present disclosure is directed to a medical device package having a flip cap and a first use indicator label that is carried by the cap.

### BACKGROUND

Intermittent catheterization is a good option for many users who suffer from a neurogenic bladder, that is, an atonic or unstable bladder associated with a neurological condition, such as diabetes, stroke, or spinal cord injury. Very often a neurogenic bladder is caused by conditions which may also result in diminished dexterity of the user.

Commonly, in intermittent catheterization, single use, individually packaged, sterile catheters are used. Such catheters are often packaged in a disposable container. Users will often prefer to return a used catheter to their purse or bag for subsequent disposal. Accordingly, the catheter package should be capable of receiving a used catheter back in the package and then being reclosed in a sealed and secured manner. Thus, a catheter package with a reliably re-closable cap would also be desired. Convenience may be further enhanced if the re-closable cap is attached to the remainder of the package, so the cap does not become misplaced during use of the catheter.

One challenge for package designers is the need to provide confirmation to the user whether or not a package has been previously opened. This challenge is heightened where the matching outside geometry of the case and cap makes it difficult to tell whether they have been previously opened, even if a tell-tale label of some sort is initially attached to the case and cap. Confirmation as to whether the seal on a package has been breached (either by intentional use of the product or by accidental opening) is highly desirable as, if the seal has been breached, then the product's sterility could be compromised. It is known to apply to the package a first use indicator label that spans the separation line of the cap and case. The label will then tear upon first opening of the package. However, when the label is fully supported by underlying structure, it has been found that the label can tear so cleanly that upon reclosing of the cap the adjoining pieces of the torn label fit back together so well as to make the tear line difficult to see. In this case there are no obvious signs that the cap has been opened, despite the initial tearing of the label.

One example of a catheter package with a first use indicator label is described in WO 2018/156589. The package described therein includes a flip cap attached to an elongated case. The case or the cap includes an indentation or chamfer over which a scored label is placed. The label spans the adjacent areas and surfaces of the cap and case such that when the package is opened (by flipping open the cap), the label is torn or otherwise ruptures to provide an indication that the package has been opened.

While the first use indicator described in WO 2018/156589 has worked satisfactorily, efforts continue to provide reliable first use indication in medical and other packaging.

### SUMMARY

There are several aspects to the subject matter disclosed herein.

In one aspect, the present disclosure is directed to a medical product package that may comprise a case for receiving a medical product, the case having an open end; a cap for sealing the open end of the case; a hinge having one end connected to the cap and a second end connected to the case; an indentation in the outer surface of the cap, the indentation having a reduced outer dimension compared to the rest of the outer surface of the cap in which it is formed; an indentation in an outer surface of the case, the indentation having a reduced outer dimension compared to the rest of the outer surface of the case in which it is formed, the indentation in the outer surface of the cap and the indentation in the outer surface of the case being adjacent to each other when the cap is in a fully closed position; and a first use indication label affixed to the cap and case and spans both the indentation in the cap and the indentation in the case, wherein the first use indication label is made of a material that does not tear upon opening of the package.

In another aspect, the present disclosure is directed to a medical product package that may comprise a case for receiving a medical product, the case having an open end; a cap for sealing the open end of the case; a hinge having one end connected to the cap and a second end connected to the case; an indentation in the outer surface of the cap, the indentation having a reduced outer dimension compared to the rest of the outer surface of the cap in which it is formed; an indentation in an outer surface of the case, the indentation having a reduced outer dimension compared to the rest of the outer surface of the case in which it is formed, the indentation in the outer surface of the cap and the indentation in the outer surface of the case being adjacent to each other when the cap is in a fully closed position; and a first use indication label affixed to the cap and case and spans both the indentation in the cap and the indentation in the case, and wherein the first use indication label remains affixed to the cap after opening the package.

In yet another aspect, the present disclosure is directed to a method implementing a first use indication label on a medical product package that may comprise providing a case for receiving a medical product, the case having an open end and an indentation in an outer surface of the case, the indentation having a reduced outer dimension compared to the rest of the outer surface of the case in which it is formed; providing a cap for sealing the open end of the case, wherein the cap comprises an indentation in an outer surface of the cap, the indentation having a reduced outer dimension compared to the rest of the outer surface of the cap in which it is formed; coupling the cap to the case using a hinge, wherein the indentation in the outer surface of the cap and the indentation in the outer surface of the case being adjacent to each other when the cap is in a fully closed position; affixing a first use indication label to the cap and the case, wherein the first use indication label spans both the indentation in the cap and the indentation in the case, wherein the first use indication label is made of a material that does not tear upon opening of the package, and wherein the first use indication label remains affixed to the cap after opening the package.

### BRIEF DESCRIPTION OF THE DRAWINGS

FIG. 1 is a perspective view of a medical product package with a first use indicator label applied thereon;

FIG. 2 is a front view of the medical product package of FIG. 1;

FIG. 3 is cross-sectional side view of the medical product package with a medical device contained therein;

FIG. 4 is an enlarged perspective view of the medical product package without the first use indicator label;

FIG. 5 is an enlarged perspective view of the medical product package with the first use indicator label placed thereon;

FIG. 6 is a partial perspective view of the medical product package with the cap opened;

FIG. 7 is a partial side view of the medical product with the cap of the package in the fully opened position;

FIG. 8 is a partial side view of the medical product with the cap moved to a partially closed position;

FIG. 9 is a partial side view of the medical product with the cap further moved to a partially closed position and the first use indicator label partially folded under the cap;

FIG. 10 is a partial perspective view of the medical product with the cap being closed by a user and the first use indicator label beginning to fold;

FIG. 11 is a partial side view of the medical product package in a fully closed position after initial opening; and

FIG. 12 is a partial front view of the medical product package in the fully closed position after an initial opening.

### DETAILED DESCRIPTION OF THE EMBODIMENTS

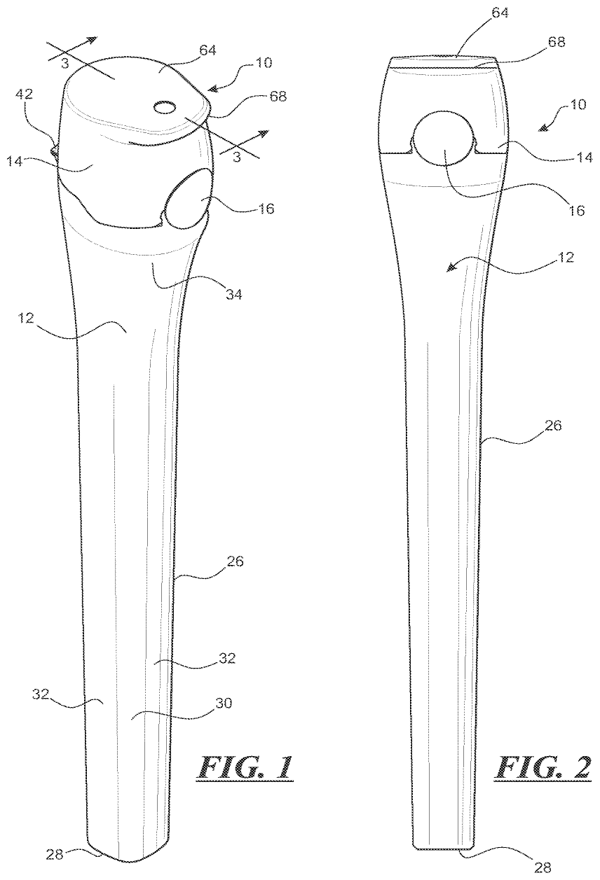

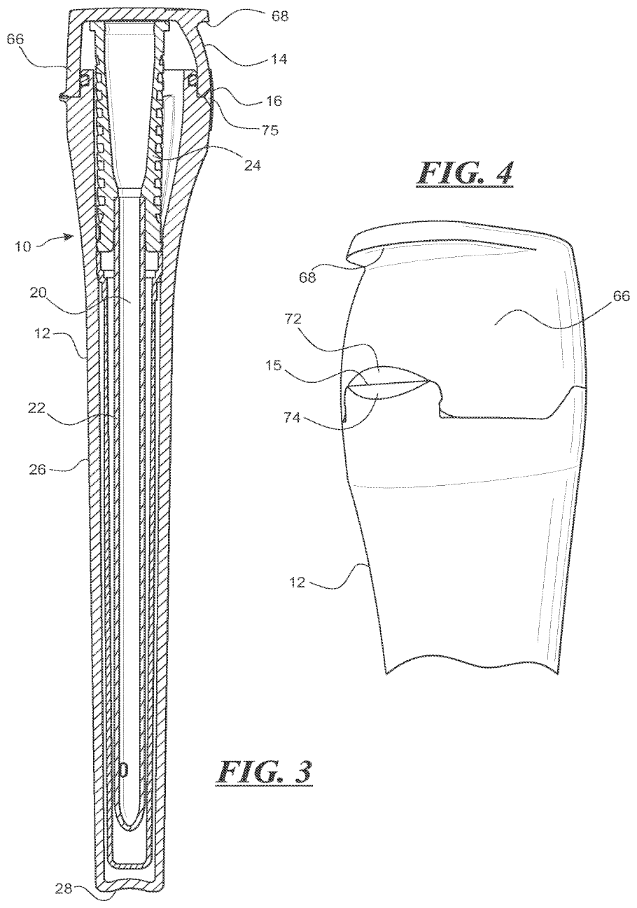

The present disclosure is directed to packages for medical products or devices such as intermittent urinary catheters. Such a package is shown generally at 10 in FIGS. 1-2. FIGS. 1-2 show the major components of the package including: a case 12, a cap 14, and a first use indicator label 16. FIG. 3 illustrates the product contained within the package 10, namely, a urinary catheter 20 which includes catheter tubing 22 and a funnel 24 attached to one (distal) end of the tubing 22. Further details of the package and package and product are described in WO 2018/156589, the contents of which are incorporated herein by reference in their entirety.

The case is preferably molded from a suitable plastic material, such as polypropylene, although other materials could be used. The case includes a hollow tubular wall 26. Where the package is for housing a catheter or other elongated product, the tubular wall 26 terminates at an end wall 28 that closes the bottom of the tubular wall. The interior surface of the tubular wall may be generally cylindrical.

The exterior surface of the tubular wall 26 may have either a cylindrical or rectangular cross-sectional shape or the cross-section could be otherwise. As shown in FIG. 1, most of the exterior surface of the tubular wall 26 has a generally rectangular cross-section with large-radiused corners 30 joining four sides 32, although as shown even the sides 32 are not perfectly flat as they have a slight curvature on their outer surfaces. Above this rectangular portion the tubular wall 26 flares outwardly somewhat at a neck portion 34 to increase the diameter of the tube.

Cap 14 is hinged to the case 12 by hinge 42 and is selectively movable by a user between a closed position and an open position. FIG. 6 shows the cap 14 in the open position. The cap 14 includes a generally cup-shaped shell having a top wall 64 joined to a generally cylindrical side wall or skirt 66. The top wall 64 and side wall 66 may merge on one side of the cap to form a lip 68 that makes it easy for a user to engage the cap with a finger or thumb. Alternately, the lip 68 can be used to assist in opening the cap by placing the lip 68 in engagement with the edge of a table or similar surface and pulling the case downwardly.

The hinge 42 as shown is preferably a living hinge of the butterfly type, but other hinge arrangements could be used. The hinge connects to the collar 36 and the cap 14 to permit selectable movement of the cap between open and closed positions.

As shown in FIGS. 1 and 2, a first use indicator label is located on the outer surface of package 10. In one embodiment, label 16 is placed over the junction of cap 14 and case 12, preferably directly opposite hinge 42. First use indicator label 16 may have a paper outer surface and an adhesive backing layer for affixation of label 16 to package 10. As will be described below, the material of label should be sufficiently strong to avoid tearing upon opening of the package (by flipping open cap 14). In addition, the adhesive on the back of label 16 should be such that the label is released from the case and remains intact during the opening sequence. The outer surface of label 16 may be blank or include a pattern or contrasting color to provide a further and clear affirmation of earlier use. The label shown in the Figures is shown as a circular label, but any shape may be used as long as label 16 spans the junction of the cap 14 and case 12 and the indentations which will now be described.

As will be appreciated from FIGS. 1 and 2 and as further described in WO 2018/156589, the outer surfaces or contours of the closed cap 14 and case 12 match one another at the junction line 15 where they meet. This provides a smooth, continuous outer contour to the package as a whole. There are no overlapping outer surfaces of the cap and case. There are no offsets, discontinuities, corners or exposed edges. This structure provides aesthetic benefits as well as the sealing benefits explained above. However, the smooth continuous outer contour also provides underlying support to the first use indication label 16.

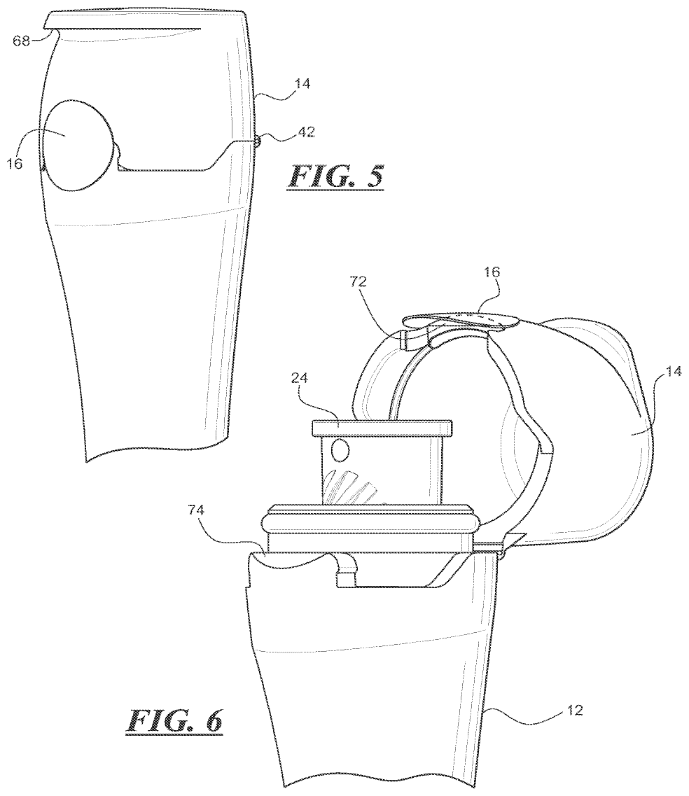

As shown in FIG. 4, an exception to the smooth continuous outer contour are an adjacent pair of indentations (or chamfers) 72 and 74 in each of the case 12 and cap 14. Each indentation is located along junction line 15 and defines an angled surface. A preferred circumferential location of the indentations 72 and 74 is centered 180° from the center of the hinge 42, i.e., at the front of the case. However, other locations for the indentations are possible. While angled chamfers as shown in FIG. 4 provide a convenient shape for creating an indentation or unsupported region under a first use indicator label, other arrangements are possible, such as a square or rectangular notch. Whatever the shape and location of the indentations, they create a depression 75 compared to the structure surrounding it and the first use indication label will span or overlie this depression or unsupported area. As seen in FIG. 5, first use indicator label 16 covers both indentations or chamfers and the depression 75 prior to the first use. The original and unaltered label shape (such as the circle in FIG. 5) provides evidence to the user that the package has not been previously opened.

In one embodiment, the size or area of the indentations 72 and 74 may be the same. Alternatively, lower indentation 74 may be greater in size (height, area) than the top indentation 72. This results in a greater surface area for the label 16 to adhere on the cap 14, rather than on the case 12, and further ensures that the label will remain with the cap 14 when opened (rather than remaining with the case 12).

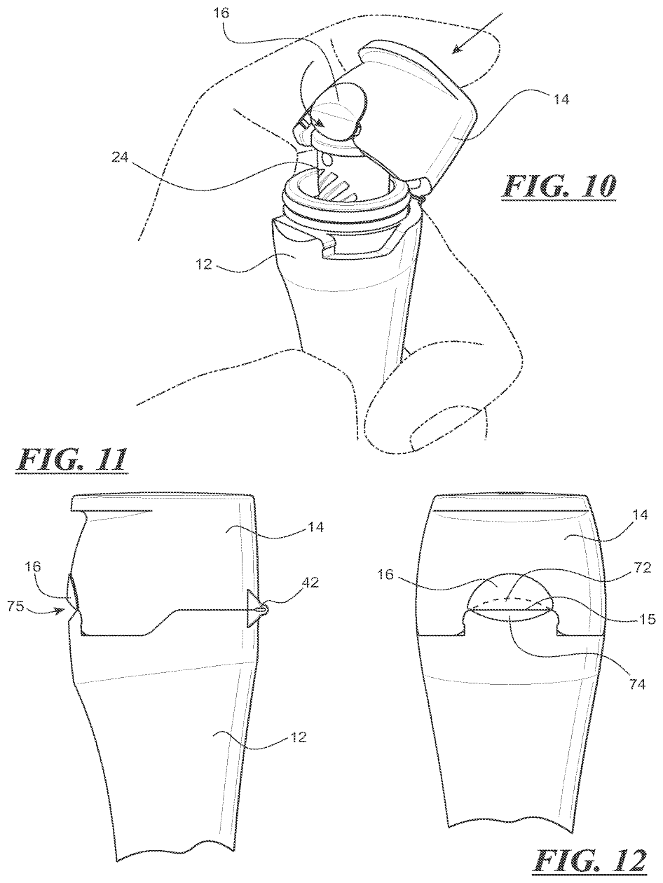

As best seen in FIGS. 8 and 10, the position of the label 16 may be raised slightly such that an increased proportion of label 16 is adhered to the cap 14 rather than the case 12. Upon opening of the case, the higher positioned label will remain adhered to the cap rather than to the case. Such placement of label 16 may be particularly advantageous when indentations 72 and 74 are of same or similar size.

Packages of the type described herein are particularly useful for housing catheters such as intermittent urinary catheters. Turning briefly to FIG. 3, an intermittent urinary catheter 20 within package 10 is shown. Catheter 20 is secured within package 10 from which it can be removed by the user at the time of use. Distal funnel 24 extends beyond the opening in case 12 (best seen in FIG. 6) where it can be gripped by the user and removed from the case.

After use of the catheter 20, it is not uncommon for users to return the used catheter 20 to its package for disposal of the catheter and the package. Faced with a package that contains a catheter, the user would like to know whether the package has been previously opened (whether or not used).

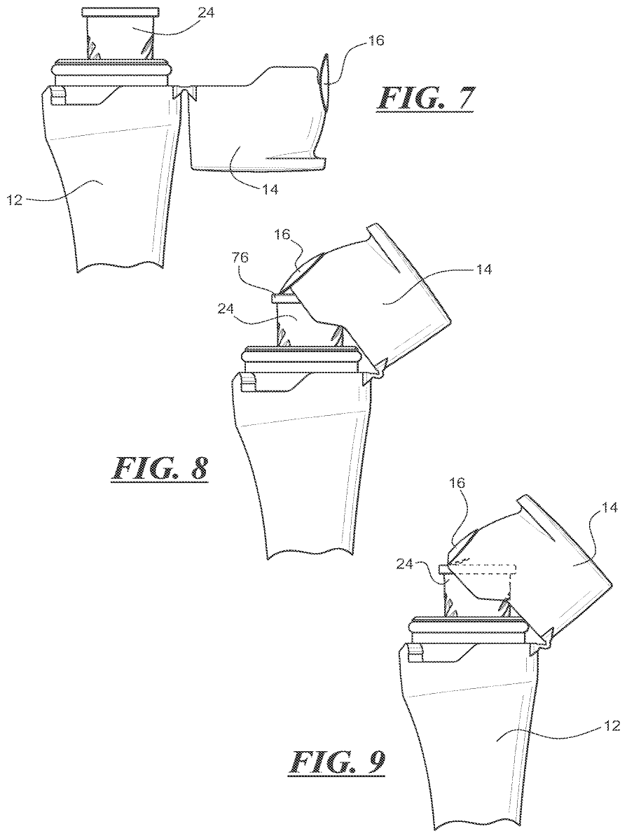

FIGS. 7-12 show the manner by which first use indication label 16 provides evidence that the package has been opened. FIG. 7 shows package 10 in its (fully) opened position with cap 14 rotated with distal funnel 24 of an intermittent urinary catheter exposed, secured within case 12 and extending beyond the opening of case 12. First use indicator label has been detached from the surface of case 12 but remains affixed to cap 14 intact with lower portion (that was previously affixed to case 12) depends from cap 14.

As shown in FIG. 8, rotating cap 14 from its open position of FIG. 7 toward a closed position causes the lower portion of label 16, and more particularly, lower edge 76 of label 16 to contact funnel 24. In this regard, funnel 24 is positioned within case 12 such that contact between the funnel (or funnel flange 78) occurs. As label 16 contacts funnel 24, the lower half of label 16 is folded inwardly over indentation 72 as shown in FIGS. 9 and 10. As cap 14 clears the funnel 24, label 16 is now folded under the lower edge of cap 14 and indentation 72 resulting in something less than the full label 16. This is perhaps best seen when comparing the label 16 in FIG. 2 to the label in FIGS. 11 and 12. The full circle label of FIG. 2 prior to initial opening becomes a recognizable and unmistakable “half-moon” label after it has been open and then reclosed.

The embodiments disclosed herein are for the purpose of providing a description of the present subject matter, and it is understood that the subject matter may be embodied in various other forms and combinations. Therefore, the specific embodiments and features disclosed herein are not to be interpreted as limiting the subject matter as defined by the accompanying claims.

1. A medical product package comprising:
a) a case for receiving a medical product, the case having an open end;
b) a cap for sealing the open end of the case;
c) a hinge having one end connected to the cap and a second end connected to the case;
d) an indentation in the outer surface of the cap, the indentation having a reduced outer dimension compared to the rest of the outer surface of the cap in which it is formed;
e) an indentation in an outer surface of the case, the indentation having a reduced outer dimension compared to the rest of the outer surface of the case in which it is formed, the indentation in the outer surface of the cap and the indentation in the outer surface of the case being adjacent to each other when the cap is in a fully closed position; and
f) a first use indication label affixed to the cap and case and spans both the indentation in the cap and the indentation in the case, wherein the first use indication label is made of a material that does not tear upon opening of the package.

2. The medical product package of claim 1 wherein the indentations are mirror images of each other.

3. The medical product package of claim 1 wherein the first use indication label is affixed to the package by an adhesive.

4. The medical product package of claim 3 wherein the first use indication label is made of a material that is releasably affixed to the case.

5. The medical package of claim 1 wherein the indentations are of different size.

6. The medical package of claim 1 wherein a larger proportion of the label is affixed to the cap than the case.

7. The medical product package of claim 1 wherein the case has a tubular wall which is closed at one end and open at the other end, the other end of the tubular wall defining a top land and an outer surface adjacent the top land, the outer surface of the tubular wall having an outer dimension.

8. The medical product package of claim 7 wherein the cap has a top wall and a side wall, the side wall defining a bottom land and an outer surface adjacent the bottom land, the outer surface of the side wall having an outer dimension that matches the outer dimension of the tubular wall.

9. The medical product package of claim 7 wherein the hinge has one end connected to the cap and a second end connected to the other end of the tubular wall, the hinge permitting selectable movement of the cap between an open position, wherein access is provided to the open end of the tubular wall, and a closed position, wherein the cap prevents access to the open end of the tubular wall.

10. The medical product package of claim 9 wherein the bottom land of the closed cap together with the top land of the case defining a junction line where the cap and case are adjacent one another when the cap is closed.

11. A medical product package comprising:
a) a case for receiving a medical product, the case having an open end;
b) a cap for sealing the open end of the case;
c) a hinge having one end connected to the cap and a second end connected to the case;
d) an indentation in the outer surface of the cap, the indentation having a reduced outer dimension compared to the rest of the outer surface of the cap in which it is formed;
e) an indentation in an outer surface of the case, the indentation having a reduced outer dimension compared to the rest of the outer surface of the case in which it is formed, the indentation in the outer surface of the cap and the indentation in the outer surface of the case being adjacent to each other when the cap is in a fully closed position; and
f) a first use indication label affixed to the cap and case and spans both the indentation in the cap and the indentation in the case, and wherein the first use indication label remains affixed to the cap after opening the package.

12. The medical product package of claim 11 wherein the first use indication label is made of a material that is releasably affixed to the case.

13. The medical product package of claim 11 wherein a larger proportion of the label is affixed to the cap than the case.

14. The medical product package of claim 11 wherein the first use indication label is affixed to the package by an adhesive.

15. A method implementing a first use indication label on a medical product package comprising:
providing a case for receiving a medical product, the case having an open end and an indentation in an outer surface of the case, the indentation having a reduced outer dimension compared to the rest of the outer surface of the case in which it is formed;
providing a cap for sealing the open end of the case, wherein the cap comprises an indentation in an outer surface of the cap, the indentation having a reduced outer dimension compared to the rest of the outer surface of the cap in which it is formed;
coupling the cap to the case using a hinge, wherein the indentation in the outer surface of the cap and the indentation in the outer surface of the case being adjacent to each other when the cap is in a fully closed position;
affixing a first use indication label to the cap and the case, wherein the first use indication label spans both the indentation in the cap and the indentation in the case, wherein the first use indication label is made of a material that does not tear upon opening of the package, and wherein the first use indication label remains affixed to the cap after opening the package.

16. The method of claim 15 wherein the indentation in an outer surface of the case and the indentation in the outer surface of the cap are mirror images of each other.

17. The method of claim 15 wherein the first use indication label is affixed to the package by an adhesive.

18. The method of claim 15 wherein the first use indication label is made of a material that is releasably affixed to the case.

19. The method of claim 15 wherein the indentation in an outer surface of the case and the indentation in the outer surface of the cap are of different size.

20. The method of claim 15 wherein a larger proportion of the first use indication label is affixed to the cap than the case.
